(19) United States
(12) Patent Application Publication
(10) Pub. No.: US 2026/0199704 A1
(43) Pub. Date: Jul. 16, 2026

(54) RADIOTHERAPY POSITIONING AND DOSE OPTIMIZATION SYSTEM BASED ON DEEP LEARNING AND MULTIMODAL IMAGE FUSION

(71) Applicant: CHANGZHOU NO.2 PEOPLE'S HOSPITAL, Changzhou (CN)

(72) Inventors: Jianlin WANG, Changzhou (CN); Chengxian MA, Changzhou (CN); Hua QIAN, Changzhou (CN); Liugang GAO, Changzhou (CN); Tian XU, Changzhou (CN)

(21) Appl. No.: 19/020,089

(22) Filed: Jan. 14, 2025

(51) Int. Cl. A61N 5/10; G16H 20/40

(52) U.S. Cl. CPC A61N 5/1031; A61N 5/1039; A61N 5/1045; A61N 5/1049; G16H 20/40

(57) ABSTRACT

The present application discloses a radiotherapy positioning and dose optimization system based on deep learning and multimodal image fusion, including a multimodal image acquisition and fusion module, a tumor positioning module, an organ motion tracking module, a dose optimization calculation module, and a quality assurance and verification module; the multimodal image acquisition and fusion module is configured to compatibly and integratively integrate data generated by a plurality of medical imaging devices, and accurately match and fuse different modal images in three-dimensional space through an advanced image alignment algorithm, to form a high-precision fusion image containing information on anatomical structure, functional metabolism, and physiological activities.

### TECHNICAL FIELD

The present application belongs to the fields of medical image processing technology, radiation therapy technology and artificial intelligence technology, and specifically relates to radiotherapy positioning and dose optimization system based on deep learning and multimodal image fusion.

### BACKGROUND

In tumor radiation therapy, accurate positioning of the tumor and the surrounding vital organs of the tumor and optimizing the radiation dose distribution accordingly are the key factors to improve the therapeutic efficacy and reduce the side effects. Currently, medical imaging technologies such as computed tomography (CT), magnetic resonance imaging (MRI), and positron emission tomography (PET) provide important anatomical and functional information for radiotherapy planning. However, single-modality images often do not provide enough information to accurately depict the full characteristics of the tumor, so that multimodal image fusion techniques are needed to integrate information from different modalities to obtain a more comprehensive image of the tumor and surrounding normal tissues.

The traditional radiotherapy positioning and dose optimization process usually requires physicians to manually outline tumor contours and organ structures based on their experience, and then physicists calculate the dose distribution, which is a time-consuming and human-induced process, resulting in potentially subjective errors in the results. And due to the high complexity and individual differences in the morphology and location of the human body and tumors, it is often difficult for traditional manual outlining methods to meet the requirements of high efficiency and accuracy.

With the development of artificial intelligence technology, especially the breakthrough progress of deep learning in the field of image recognition and analysis, the application of deep learning in high-precision medical image analysis has become possible. Deep learning models can learn a large amount of medical image data, automatically extract complex features, and achieve accurate identification and segmentation of tumors and their surrounding structures. In addition, deep learning algorithms can improve their accuracy and robustness in the process of continuous learning, thus adapting to a variety of complex and changing treatment scenarios, such that it proposes a radiotherapy positioning and dose optimization system based on deep learning and multimodal image fusion.

### SUMMARY

It is an object of the present application to provide a radiotherapy positioning and dose optimization system based on deep learning and multimodal image fusion, in order to solve the above problems raised in the background technology.

In order to realize the above object, the present application provides the following technical solution:
a radiotherapy positioning and dose optimization system based on deep learning and multimodal image fusion, comprising a multimodal image acquisition and fusion module, a tumor positioning module, an organ motion tracking module, a dose optimization calculation module, and a quality assurance and verification module;
the multimodal image acquisition and fusion module is configured to compatibly and integratively integrate data generated by a plurality of medical imaging devices, and accurately match and fuse different modal images in three-dimensional space through an advanced image alignment algorithm, to form a high-precision fusion image containing information on anatomical structure, functional metabolism, and physiological activities;
the multimodal image acquisition and fusion module includes a multimodal image acquisition unit, a multimodal image processing unit, an image alignment unit, and a multimodal information fusion unit;
the multimodal image acquisition unit is configured to collect different types of medical image data;
the multimodal image processing unit is configured to standardize the medical image data to ensure quality and comparability of different modal images;
the image alignment unit is configured for spatial alignment of the different modal images using a non-rigid alignment algorithm based on feature points, image similarity, intensity information, geometric transformation or deep learning, and for accurately aligning the medical image data at different time points using a four-dimensional image alignment method for dynamic organs;
the multimodal information fusion unit is configured to use an image fusion algorithm to fuse the information of the multimodal images into a comprehensive image that reveals multifaceted information about the anatomical structure, the functional metabolism, and hemodynamics of a lesion site, and to train a neural network model to jointly analyze and fuse the multimodal data, to generate a fused image with richer diagnostic information;
the tumor positioning module is configured to use a deep learning architecture to achieve automated, high-precision segmentation and positioning of the tumor and substructures of the tumor by learning from a large number of annotated samples, while considering respiratory gating image data to capture dynamic changes of the tumor in different respiratory stages;
the tumor positioning module includes a data preprocessing unit, a feature extraction unit, and a positioning network structure unit;
the data preprocessing unit is configured for positioning of vision and preprocessing of received image or point cloud data;
the feature extraction unit is configured to extract high-level semantic features and local features from an original image or depth data using a convolutional neural network;
the positioning network structure unit is configured to use different network structure designs to output bounding boxes and categories simultaneously during target detection or to accurately estimate location coordinates of the tumor using a regression network;
the organ motion tracking module is configured to integrate real-time or prospectively predicted organ motion models, quantify and predict a motion trajectory of the tumor during treatment by a four-dimensional computerized tomography (CT) or other dynamic imaging data, and dynamically position dynamic intensity-modulated radiation therapy and stereotactic radiation therapy in real time;
the dose optimization calculation module is configured to use evolutionary algorithms, a Monte Carlo simulation, a linear programming or other optimization means to construct a complex dose distribution model that satisfies a treatment target based on deep learning positioning results and organ motion information, to maximize a coverage of a tumor target area while minimizing an irradiated dose of surrounding critical organs;
steps of the dose optimization calculation module for optimizing the dose are:
S1, definition of a treatment goal: specifying a main goal of a treatment plan, comprising tumor dose coverage, and a maximum permissible dose to normal tissues around the target area;
S2, construction of dose calculation modeling: applying physics-based dose calculation engines, comprising a Monte Carlo method, a collapsed cone convolution or pencil beam algorithms, to accurately calculate a distribution of radiation penetration and deposition in human tissues;
S3, optimization variable setting: setting optimization variables affecting a radiation therapy dose distribution, comprising multileaf collimator, blade position, beam direction and dose rate;
S4, constraint setting: setting constraints based on clinical needs and patient anatomy, comprising a minimum acceptable dose for the tumor target area and a maximum tolerated dose for critical organs;
S5, optimization algorithm selection: adopting a suitable optimization algorithm, comprising linear programming, integer programming, genetic algorithm, simulated annealing algorithm, particle swarm optimization algorithm, or deep reinforcement learning, to find an optimal dose distribution that satisfies all the constraints;
the quality assurance and validation module is configured to validate and adjust the finalized radiation treatment plan through a physical or virtual dose validation method to ensure that an actual executed dose distribution is consistent with the treatment plan;
the validation method includes an electron density mapping, a body mode measurement, and a dose reconstruction technique.

In one embodiment, the medical image data includes CT, magnetic resonance imaging (MRI), positron emission tomography (PET), single photon emission computed tomography (SPECT), ultrasound, and digital subtraction angiography (DSA) data; and
the standardized processing comprises preprocessing of gray scale correction, noise removal, and smoothing.

In one embodiment, the multimodal image acquisition and fusion module further includes a data interaction and visualization unit;
the data interaction and visualization unit is configured to design a user-friendly interactive interface for the doctor to view the original image, the aligned image, and the fused image, and to support a multi-window contrast and three-dimensional rendering, and to display a linkage of the image data with information on dose distribution and the treatment plan.

In one embodiment, the tumor positioning module further includes a positioning algorithm unit, a loss function design unit, a training unit, and a post-processing unit;
the positioning algorithm unit is configured to combine a deep learning method to construct an environment map while real-time positioning is performed and to combine a plurality of sensor data to perform a signal fingerprint matching and a signal fingerprinting matching by a deep learning model;
the loss function design unit is configured to measure a difference between a predicted position and an actual position, comprising an euclidean distance, a IoU index, and improve a positioning accuracy of the model by optimizing a loss function;
the training unit is configured to train using a large-scale dataset with real geo-tagging, and the dataset includes static images, video sequences, continuous sensor readings; and
the post-processing unit is configured to perform further post-processing steps of filtering, smoothing, and confidence assessment on the data to improve positioning accuracy and reliability.

In one embodiment, the organ motion tracking module is configured to achieve tracking of dynamic changes of the tumor by a three dimensional (3D) convolutional neural network, a long and short-term memory network, and an adaptive optimization algorithm.

In one embodiment, the 3D convolutional neural network extracts image features by performing convolution operations in a three dimensional space, including:

$$( {O}_{i , j , k} ) = {\sum }_{m , n , p , q} {I}_{i + m - 1 , j + n - 1 , k + p - 1} \cdot {W}_{m , n , p , q} + b ;$$

wherein O_{i,j,k} is a pixel value of an output feature map in an ith row, jth column, and kth layer; I_{i+m-1,j+n-1,k+p-1} is a pixel value in an input image cube, at a corresponding position, wherein m, n, and p are offsets of a convolution kernel in each dimension; and W_{m,n,p,q} is a weight of the convolution kernel in a mth row, nth column, pth layer, and qth channel, and b is a bias term.

In one embodiment, a long and short-term memory network is configured to process sequential data to capture time dependency, including:

$${f}_{t} = σ ( {W}_{f} \cdot [ {h}_{t - 1} , {x}_{t} ] + {b}_{f} )$$

$${i}_{t} = σ ( {W}_{i} \cdot [ {h}_{t - 1} , {x}_{t} ] + {b}_{i} )$$

$${o}_{t} = σ ( {W}_{o} \cdot [ {h}_{t - 1} , {x}_{t} ] + {b}_{o} )$$

$${c}_{t} = {f}_{t} ⊙ {c}_{t - 1} + {i}_{t} ⊙ tanh ( {W}_{c} \cdot [ {h}_{t - 1} , {x}_{t} ] + {b}_{c} )$$

$${h}_{t} = {o}_{t} ⊙ tanh ( {c}_{t} ) ;$$

where fₜ, iₜ, oₜ represent activation values of a forgetting gate, an input gate, and an output gate, respectively, at time step t, which are between 0 and 1, and determine how the information is forgotten, updated, or output; cₜ is a cellular state, which preserves the long-term dependency information from the past to a current time step; hₜ is a hidden state, which is an output of the current time step, and combines forgotten information with new input information; and σ is a sigmoid function for squeezing an input value to between (0, 1) and is commonly used in gating mechanisms; tanh is a hyperbolic tangent function for squeezing the input to (−1, 1) and is commonly used in state updating; W_{f}, Wᵢ, Wₒ, W_{c} are weight matrices corresponding to the forgetting gate, the input gate, the output gate, and a unitary state updating, respectively; b_{f}, bᵢ, bₒ, b_{c} are bias terms corresponding to each gate and the state updating; [hₜ₋₁, xₜ] is a splice vector containing a hidden state hₜ₋₁ of a previous time step and an input xₜ of a current time step; and ⊚ denotes an elementwise multiplication operation, i.e., multiplication of corresponding elements of two same shaped arrays.

In one embodiment, the adaptive optimization algorithm is configured to optimize network weights, including:

$${m}_{t} = {β}_{1} {m}_{t - 1} + ( 1 - {β}_{1} ) {g}_{t}$$

$${v}_{t} = {β}_{2} {v}_{t - 1} + ( 1 - {β}_{2} ) {g}_{t}^{2}$$

$${\hat{m}}_{t} = \frac{{m}_{t}}{1 - {β}_{1}^{t}}$$

$${\hat{v}}_{t} = \frac{{v}_{t}}{1 - {β}_{2}^{t}}$$

$${θ}_{t} = {θ}_{t - 1} - α \frac{{\hat{m}}_{t}}{\sqrt{{\hat{v}}_{t}} + ε}$$

where mₜ, vₜ are a first-order moment estimate (momentum) and a second-order moment estimate (variance) at a tth iteration, respectively; β₁, β₂ are hyperparameters of Adam's optimizer, which control decay rates of the first-order moment estimate and the second-order moment estimate, respectively; gₜ is a gradient at the tth iteration; \hat{m}ₜ, \hat{v}ₜ is a corrected (bias-corrected) first-order moment estimate and second-order moment estimate, which are configured to solve an initial bias problem; α is a learning rate, which determines a step size updated every time; ε is a constant to avoid division by zero.

In one embodiment, steps of the dose optimization calculation module for optimizing the dose further includes:
S6, dose distribution evaluation: after the optimization process, evaluating the generated dose distribution, comprising a dose-volume histogram analysis, to evaluate whether a target region and critical organs have achieved a predetermined dosimetric target;
S7, multiple iterations and optimization: the optimization calculation module performing multiple iterations to gradually approximate an ideal dose distribution, wherein each iteration is corrected and refined based on a previous result.

In one embodiment, the particle swarm optimization algorithm specifically includes:
velocity update:

$${v}_{id} ( t + 1 ) = w \cdot {v}_{id} ( t ) + {c}_{1} {r}_{1} ( {pbest}_{id} - {x}_{id} ( t ) ) + {c}_{2} {r}_{2} ( gbest - {x}_{id} ( t ) ) ;$$

position update:

$${x}_{id} ( t + 1 ) = {x}_{id} ( t ) + {v}_{id} ( t + 1 ) ;$$

where, v_{id} (t) is a velocity vector of a particle i at the tth iteration, which represents a velocity of the particle i in each dimension of a search space; x_{id} (t) is a position vector of the particle i at the tth iteration, which represents a current position of the particle i in the search space; pbest_{id} is a personal optimal position of the particle i, i.e., an optimal solution found by the particle i during a search process, the gbest is a global optimal position, i.e., a current optimal solution found in a whole swarm; w is an inertia weight for balancing a degree of memory of the particle's own speed and a degree of following the optimal information during the search process, and the inertia weight is gradually reduced with the number of iterations to improve a convergence of the algorithm; c₁ and c₂ are acceleration constants, which control the extent to which the particle adjust its velocity toward an individual optimal solution and a global optimal solution for a group of which the individual is a member; r₁ and r₂ are random numbers, which are randomly generated from [0, 1] at each iteration and are configured to introduce a random factor to avoid the algorithm from converging to the local optimal solution prematurely;
the simulated annealing algorithm specifically includes:

$$P ( accept ) = min ( 1 , {e}^{- \frac{Δ E}{T}} )$$

$$Δ E = {E}_{new} - {E}_{current} ;$$

where P(accept) denotes a probability of a worse new solution; E_{new} denotes an energy of a new state; E_{current} denotes an energy of a current state; ΔE denotes an energy difference, if ΔE is negative, a new solution is superior to a current solution; if ΔE is positive, the new solution is inferior to the current solution; T denotes a simulated temperature.

Compared with the related art, the beneficial effects of the present application are:
(1) By setting up a multimodal image acquisition and fusion module and a tumor positioning module, the present application significantly improves the positioning accuracy of the tumor and its microenvironment through multimodal image fusion and deep learning technology, and reduces the uncertainty brought about by human misjudgment;
(2) By setting up an organ movement tracking module and a dose optimization calculation module, the present application can accurately track organ movement and optimize dose distribution, which helps to avoid or mitigate damage to vital organs due to radiation, improve the quality of life and treatment safety of patients, and enable each patient's radiation treatment plan to be tailored to his or her unique medical condition, thus enhancing the personalization and pertinence of treatment;
(3) Through the quality assurance and validation module, the present application enables an automated and intelligent process to greatly shorten the design and validation time of the radiation treatment plan, and improves clinical work efficiency.

### BRIEF DESCRIPTION OF THE DRAWINGS

FIG. 1 is a structural block diagram of the present application.

FIG. 2 is a structural block diagram of a multimodal image acquisition and fusion module in the present application.

FIG. 3 is a structural block diagram of a tumor positioning module in the present application.

### DETAILED DESCRIPTION OF THE EMBODIMENTS

The technical solutions in the embodiments of the present application will be clearly and completely described below in conjunction with the accompanying drawings in the embodiments of the present application, and it is clear that the described embodiments are only a part of the embodiments of the present application and not all of the embodiments. Based on the embodiments in the present application, all other embodiments obtained by those skilled in the art without making creative labor fall within the scope of the present application.

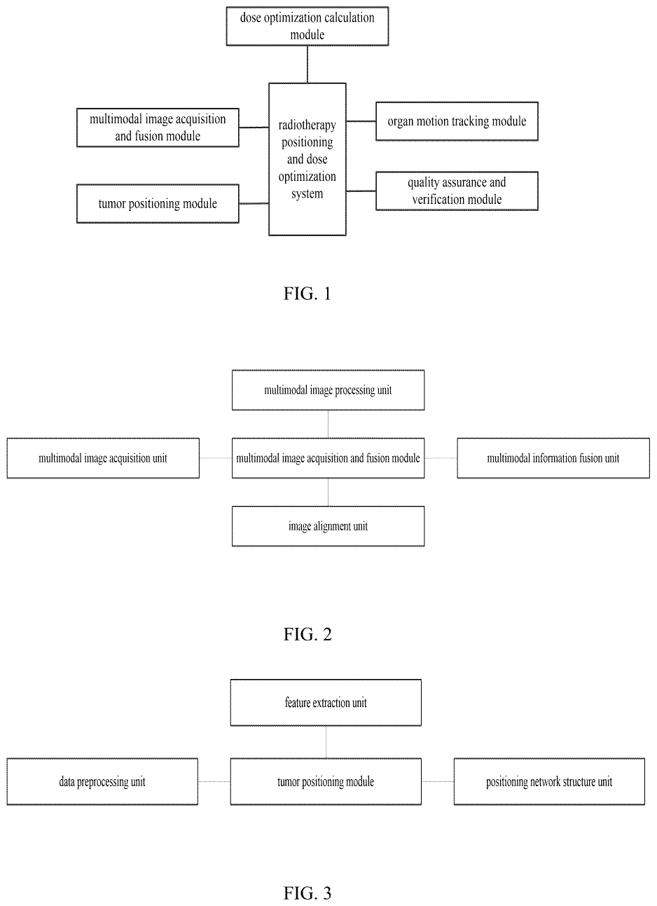

Referring to FIGS. 1-FIG. 3, the present application provides a technical solution

A radiotherapy positioning and dose optimization system based on deep learning and multimodal image fusion, comprising a multimodal image acquisition and fusion module, a tumor positioning module, an organ motion tracking module, a dose optimization calculation module, and a quality assurance and verification module;
wherein the multimodal image acquisition and fusion module is configured to compatibly and integratively integrate data generated by a plurality of medical imaging devices, and accurately match and fuse different modal images in three-dimensional space through an advanced image alignment algorithm, to form a high-precision fusion image containing information on anatomical structure, functional metabolism, and physiological activities;
wherein the multimodal image acquisition and fusion module comprises a multimodal image acquisition unit, a multimodal image processing unit, an image alignment unit, and a multimodal information fusion unit;
the multimodal image acquisition unit is configured to collect different types of medical image data;
the multimodal image processing unit is configured to standardize the medical image data to ensure quality and comparability of different modal images;
the image alignment unit is configured for spatial alignment of the different modal images using a non-rigid alignment algorithm based on feature points, image similarity, intensity information, geometric transformation or deep learning, and for accurately aligning the medical image data at different time points using a four-dimensional image alignment method for dynamic organs;
the multimodal information fusion unit is configured to use an image fusion algorithm to fuse the information of the multimodal images into a comprehensive image that reveals multifaceted information about the anatomical structure, the functional metabolism, and hemodynamics of a lesion site, and to train a neural network model to jointly analyze and fuse the multimodal data, to generate a fused image with richer diagnostic information;
the tumor positioning module is configured to use a deep learning architecture to achieve automated, high-precision segmentation and positioning of the tumor and substructures of the tumor by learning from a large number of annotated samples, while considering respiratory gating image data to capture dynamic changes of the tumor in different respiratory stages;
the tumor positioning module includes a data preprocessing unit, a feature extraction unit, and a positioning network structure unit;
the data preprocessing unit is configured for positioning of vision and preprocessing of received image or point cloud data;
the feature extraction unit is configured to extract high-level semantic features and local features from an original image or depth data using a convolutional neural network;
the positioning network structure unit is configured to use different network structure designs to output bounding boxes and categories simultaneously during target detection or to accurately estimate location coordinates of the tumor using a regression network;
the organ motion tracking module is configured to integrate real-time or prospectively predicted organ motion models, quantify and predict a motion trajectory of the tumor during treatment by a four-dimensional computerized tomography (CT) or other dynamic imaging data, and dynamically position dynamic intensity-modulated radiation therapy and stereotactic radiation therapy in real time;
the dose optimization calculation module is configured to use evolutionary algorithms, a Monte Carlo simulation, a linear programming or other optimization means to construct a complex dose distribution model that satisfies a treatment target based on deep learning positioning results and organ motion information, to maximize a coverage of a tumor target area while minimizing an irradiated dose of surrounding critical organs;
steps of the dose optimization calculation module for optimizing the dose are:
S1, definition of a treatment goal: specifying a main goal of a treatment plan, comprising tumor dose coverage, and a maximum permissible dose to normal tissues around the target area;
S2, construction of dose calculation modeling: applying physics-based dose calculation engines, comprising a Monte Carlo method, a collapsed cone convolution or pencil beam algorithms, to accurately calculate a distribution of radiation penetration and deposition in human tissues;
S3, optimization variable setting: setting optimization variables affecting a radiation therapy dose distribution, comprising multileaf collimator, blade position, beam direction and dose rate;
S4, constraint setting: setting constraints based on clinical needs and patient anatomy, comprising a minimum acceptable dose for the tumor target area and a maximum tolerated dose for critical organs;
S5, optimization algorithm selection: adopting a suitable optimization algorithm, comprising linear programming, integer programming, genetic algorithm, simulated annealing algorithm, particle swarm optimization algorithm, or deep reinforcement learning, to find an optimal dose distribution that satisfies all the constraints;
the quality assurance and validation module is configured to validate and adjust the finalized radiation treatment plan through a physical or virtual dose validation method to ensure that an actual executed dose distribution is consistent with the treatment plan;
the validation method includes an electron density mapping, a body mode measurement, and a dose reconstruction technique.

In one embodiment, the medical image data includes CT, magnetic resonance imaging (MRI), positron emission tomography (PET), single photon emission computed tomography (SPECT), ultrasound, and digital subtraction angiography (DSA) data; and the standardized processing includes preprocessing of gray scale correction, noise removal, and smoothing.

In one embodiment, the multimodal image acquisition and fusion module further comprises a data interaction and visualization unit;
the data interaction and visualization unit is configured to design a user-friendly interactive interface for the doctor to view the original image, the aligned image, and the fused image, and to support a multi-window contrast and three-dimensional rendering, and to display a linkage of the image data with information on dose distribution and the treatment plan.
In one embodiment, the tumor positioning module further includes a positioning algorithm unit, a loss function design unit, a training unit, and a post-processing unit;
the positioning algorithm unit is configured to combine a deep learning method to construct an environment map while real-time positioning is performed and to combine a plurality of sensor data to perform a signal fingerprint matching and a signal fingerprinting matching by a deep learning model;
the loss function design unit is configured to measure a difference between a predicted position and an actual position, including an euclidean distance, a IoU index, and improve a positioning accuracy of the model by optimizing a loss function;
the training unit is configured to train using a large-scale dataset with real geo-tagging, and wherein the dataset comprises static images, video sequences, continuous sensor readings; and
the post-processing unit is configured to perform further post-processing steps of filtering, smoothing, and confidence assessment on the data to improve positioning accuracy and reliability.
In one embodiment, the organ motion tracking module is configured to achieve tracking of dynamic changes of the tumor by a three dimensional (3D) convolutional neural network, a long and short-term memory network, and an adaptive optimization algorithm.

In one embodiment, the 3D convolutional neural network extracts image features by performing convolution operations in a three dimensional space, comprising:

$$( {O}_{i , j , k} ) = {\sum }_{m , n , p , q} {I}_{i + m - 1 , j + n - 1 , k + p - 1} \cdot {W}_{m , n , p , q} + b ;$$

wherein O_{i,j,k} is a pixel value of an output feature map in an ith row, jth column, and kth layer; I_{i+m-1,j+n-1,k+p-1} is a pixel value in an input image cube, at a corresponding position, wherein m, n, and p are offsets of a convolution kernel in each dimension; and W_{m,n,p,q} is a weight of the convolution kernel in a mth row, nth column, pth layer, and qth channel, and b is a bias term.

In one embodiment, a long and short-term memory network is configured to process sequential data to capture time dependency, including:

$${f}_{t} = σ ( {W}_{f} \cdot [ {h}_{t - 1} , {x}_{t} ] + {b}_{f} )$$

$${i}_{t} = σ ( {W}_{i} \cdot [ {h}_{t - 1} , {x}_{t} ] + {b}_{i} )$$

$${o}_{t} = σ ( {W}_{o} \cdot [ {h}_{t - 1} , {x}_{t} ] + {b}_{o} )$$

$${c}_{t} = {f}_{t} ⊙ {c}_{t - 1} + {i}_{t} ⊙ tanh ( {W}_{c} \cdot [ {h}_{t - 1} , {x}_{t} ] + {b}_{c} )$$

$${h}_{t} = {o}_{t} ⊙ tanh ( {c}_{t} ) ;$$

where fₜ, iₜ, oₜ represent activation values of a forgetting gate, an input gate, and an output gate, respectively, at time step t, which are between 0 and 1, and determine how the information is forgotten, updated, or output; cₜ is a cellular state, which preserves the long-term dependency information from the past to a current time step; hₜ is a hidden state, which is an output of the current time step, and combines forgotten information with new input information; and σ is a sigmoid function for squeezing an input value to between (0, 1) and is commonly used in gating mechanisms; tanh is a hyperbolic tangent function for squeezing the input to (−1, 1) and is commonly used in state updating; W_{f}, Wᵢ, Wₒ, W_{c} are weight matrices corresponding to the forgetting gate, the input gate, the output gate, and a unitary state updating, respectively; b_{f}, bᵢ, bₒ, b_{c} are bias terms corresponding to each gate and the state updating; [hₜ₋₁, xₜ] is a splice vector containing a hidden state hₜ₋₁ of a previous time step and an input xₜ of a current time step; and ⊚ denotes an elementwise multiplication operation, i.e., multiplication of corresponding elements of two same shaped arrays.

In one embodiment, the adaptive optimization algorithm is configured to optimize network weights, including:

$${m}_{t} = {β}_{1} {m}_{t - 1} + ( 1 - {β}_{1} ) {g}_{t}$$

$${v}_{t} = {β}_{2} {v}_{t - 1} + ( 1 - {β}_{2} ) {g}_{t}^{2}$$

$${\hat{m}}_{t} = \frac{{m}_{t}}{1 - {β}_{1}^{t}}$$

$${\hat{v}}_{t} = \frac{{v}_{t}}{1 - {β}_{2}^{t}}$$

$${θ}_{t} = {θ}_{t - 1} - α \frac{{\hat{m}}_{t}}{\sqrt{{\hat{v}}_{t}} + ε}$$

where mₜ, vₜ are a first-order moment estimate (momentum) and a second-order moment estimate (variance) at a tth iteration, respectively; β₁, β₂ are hyperparameters of Adam's optimizer, which control decay rates of the first-order moment estimate and the second-order moment estimate, respectively; gₜ is a gradient at the tth iteration; \hat{m}ₜ, \hat{v}ₜ, is a corrected (bias-corrected) first-order moment estimate and second-order moment estimate, which are configured to solve an initial bias problem; α is a learning rate, which determines a step size updated every time; ε is a constant to avoid division by zero.

In one embodiment, steps of the dose optimization calculation module for optimizing the dose further includes:
S6, dose distribution evaluation: after the optimization process, evaluating the generated dose distribution, comprising a dose-volume histogram analysis, to evaluate whether a target region and critical organs have achieved a predetermined dosimetric target;
S7, multiple iterations and optimization: the optimization calculation module performing multiple iterations to gradually approximate an ideal dose distribution, wherein each iteration is corrected and refined based on a previous result.

In one embodiment, the particle swarm optimization algorithm specifically includes:
velocity update:

$${v}_{id} ( t + 1 ) = w \cdot {v}_{id} ( t ) + {c}_{1} {r}_{1} ( {pbest}_{id} - {x}_{id} ( t ) ) + {c}_{2} {r}_{2} ( gbest - {x}_{id} ( t ) ) ;$$

position update:

$${x}_{id} ( t + 1 ) = {x}_{id} ( t ) + {v}_{id} ( t + 1 ) ;$$

where, v_{id} (t) is a velocity vector of a particle i at the tth iteration, which represents a velocity of the particle i in each dimension of a search space; x_{id} (t) is a position vector of the particle i at the tth iteration, which represents a current position of the particle i in the search space; pbest_{id} is a personal optimal position of the particle i, i.e., an optimal solution found by the particle i during a search process, the gbest is a global optimal position, i.e., a current optimal solution found in a whole swarm; w is an inertia weight for balancing a degree of memory of the particle's own speed and a degree of following the optimal information during the search process, and the inertia weight is gradually reduced with the number of iterations to improve a convergence of the algorithm; c₁ and c₂ are acceleration constants, which control the extent to which the particle adjust its velocity toward an individual optimal solution and a global optimal solution for a group of which the individual is a member; r₁ and r₂ are random numbers, which are randomly generated from [0, 1] at each iteration and are configured to introduce a random factor to avoid the algorithm from converging to the local optimal solution prematurely;
the simulated annealing algorithm specifically includes:

$$P ( accept ) = min ( 1 , {e}^{- \frac{Δ E}{T}} )$$

$$Δ E = {E}_{new} - {E}_{current} ;$$

where P(accept) denotes a probability of a worse new solution; E_{new} denotes an energy of a new state; E_{current} denotes an energy of a current state; ΔE denotes an energy difference, if ΔE is negative, a new solution is superior to a current solution; if ΔE is positive, the new solution is inferior to the current solution; T denotes a simulated temperature.

Principle and advantages of the present application:
By setting a multimodal image acquisition and fusion module and a tumor positioning module, the present application significantly improves the positioning accuracy of the tumor and its microenvironment through multimodal image fusion and deep learning technology, and reduces the uncertainty brought about by human misjudgment; by setting an organ motion tracking module and a dose optimization calculation module, the present application can accurately track the organ motion and optimize the dose distribution, which can help to avoid or mitigate the damage of vital organs caused by radiation, improving the quality of life of the patient and the safety of the treatment, and enabling the radiation treatment plan of each patient to be tailored based on his or her unique condition, enhancing the personalization and targeting of the treatment; and through the quality assurance and validation module, the present application enables an automated and intelligent process to greatly shorten the time for the design and validation of the radiation treatment plan, and improving the efficiency of the clinical work.

Although embodiments of the present application have been shown and described, it will be appreciated by those skilled in the art that a variety of changes, modifications, substitutions, and variations may be made to these embodiments without departing from the principles and spirit of the present application, and the scope of the present application is limited by the appended claims and their equivalents.

What is claimed is:

1. A radiotherapy positioning and dose optimization system based on deep learning and multimodal image fusion, comprising a multimodal image acquisition and fusion module, a tumor positioning module, an organ motion tracking module, a dose optimization calculation module, and a quality assurance and verification module;
wherein the multimodal image acquisition and fusion module is configured to compatibly and integratively integrate data generated by a plurality of medical imaging devices, and accurately match and fuse different modal images in three-dimensional space through an advanced image alignment algorithm, to form a high-precision fusion image containing information on anatomical structure, functional metabolism, and physiological activities;
wherein the multimodal image acquisition and fusion module comprises a multimodal image acquisition unit, a multimodal image processing unit, an image alignment unit, and a multimodal information fusion unit;
the multimodal image acquisition unit is configured to collect different types of medical image data;
the multimodal image processing unit is configured to standardize the medical image data to ensure quality and comparability of different modal images;
the image alignment unit is configured for spatial alignment of the different modal images using a non-rigid alignment algorithm based on feature points, image similarity, intensity information, geometric transformation or deep learning, and for accurately aligning the medical image data at different time points using a four-dimensional image alignment method for dynamic organs;
the multimodal information fusion unit is configured to use an image fusion algorithm to fuse the information of the multimodal images into a comprehensive image that reveals multifaceted information about the anatomical structure, the functional metabolism, and hemodynamics of a lesion site, and to train a neural network model to jointly analyze and fuse the multimodal data, to generate a fused image with richer diagnostic information;
the tumor positioning module is configured to use a deep learning architecture to achieve automated, high-precision segmentation and positioning of the tumor and substructures of the tumor by learning from a large number of annotated samples, while considering respiratory gating image data to capture dynamic changes of the tumor in different respiratory stages;
wherein the tumor positioning module comprises a data preprocessing unit, a feature extraction unit, and a positioning network structure unit;
the data preprocessing unit is configured for positioning of vision and preprocessing of received image or point cloud data;
the feature extraction unit is configured to extract high-level semantic features and local features from an original image or depth data using a convolutional neural network;
the positioning network structure unit is configured to use different network structure designs to output bounding boxes and categories simultaneously during target detection or to accurately estimate location coordinates of the tumor using a regression network;
the organ motion tracking module is configured to integrate real-time or prospectively predicted organ motion models, quantify and predict a motion trajectory of the tumor during treatment by a four-dimensional computerized tomography (CT) or other dynamic imaging data, and dynamically position dynamic intensity-modulated radiation therapy and stereotactic radiation therapy in real time;
the dose optimization calculation module is configured to use evolutionary algorithms, a Monte Carlo simulation, a linear programming or other optimization means to construct a complex dose distribution model that satisfies a treatment target based on deep learning positioning results and organ motion information, to maximize a coverage of a tumor target area while minimizing an irradiated dose of surrounding critical organs;
wherein steps of the dose optimization calculation module for optimizing the dose are:
S1, definition of a treatment goal: specifying a main goal of a treatment plan, comprising tumor dose coverage, and a maximum permissible dose to normal tissues around the target area;
S2, construction of dose calculation modeling: applying physics-based dose calculation engines, comprising a Monte Carlo method, a collapsed cone convolution or pencil beam algorithms, to accurately calculate a distribution of radiation penetration and deposition in human tissues;
S3, optimization variable setting: setting optimization variables affecting a radiation therapy dose distribution, comprising multileaf collimator, blade position, beam direction and dose rate;
S4, constraint setting: setting constraints based on clinical needs and patient anatomy, comprising a minimum acceptable dose for the tumor target area and a maximum tolerated dose for critical organs;
S5, optimization algorithm selection: adopting a suitable optimization algorithm, comprising linear programming, integer programming, genetic algorithm, simulated annealing algorithm, particle swarm optimization algorithm, or deep reinforcement learning, to find an optimal dose distribution that satisfies all the constraints;
the quality assurance and validation module is configured to validate and adjust the finalized radiation treatment plan through a physical or virtual dose validation method to ensure that an actual executed dose distribution is consistent with the treatment plan;
wherein the validation method comprises an electron density mapping, a body mode measurement, and a dose reconstruction technique.

2. The radiotherapy positioning and dose optimization system based on deep learning and multimodal image fusion according to claim 1, wherein the medical image data comprises CT, magnetic resonance imaging (MRI), positron emission tomography (PET), single photon emission computed tomography (SPECT), ultrasound, and digital subtraction angiography (DSA) data; and
the standardized processing comprises preprocessing of gray scale correction, noise removal, and smoothing.

3. The radiotherapy positioning and dose optimization system based on deep learning and multimodal image fusion based on claim 2, wherein the multimodal image acquisition and fusion module further comprises a data interaction and visualization unit;
the data interaction and visualization unit is configured to design a user-friendly interactive interface for the doctor to view the original image, the aligned image, and the fused image, and to support a multi-window contrast and three-dimensional rendering, and to display a linkage of the image data with information on dose distribution and the treatment plan.

4. The radiotherapy positioning and dose optimization system based on deep learning and multimodal image fusion according to claim 1, wherein the tumor positioning module further comprises a positioning algorithm unit, a loss function design unit, a training unit, and a post-processing unit;
the positioning algorithm unit is configured to combine a deep learning method to construct an environment map while real-time positioning is performed and to combine a plurality of sensor data to perform a signal fingerprint matching and a signal fingerprinting matching by a deep learning model;
the loss function design unit is configured to measure a difference between a predicted position and an actual position, comprising an euclidean distance, a IoU index, and improve a positioning accuracy of the model by optimizing a loss function;
the training unit is configured to train using a large-scale dataset with real geo-tagging, and wherein the dataset comprises static images, video sequences, continuous sensor readings; and
the post-processing unit is configured to perform further post-processing steps of filtering, smoothing, and confidence assessment on the data to improve positioning accuracy and reliability.

5. The radiotherapy positioning and dose optimization system based on deep learning and multimodal image fusion according to claim 1, wherein the organ motion tracking module is configured to achieve tracking of dynamic changes of the tumor by a three dimensional (3D) convolutional neural network, a long and short-term memory network, and an adaptive optimization algorithm.

6. The radiotherapy positioning and dose optimization system based on deep learning and multimodal image fusion according to claim 5, wherein the 3D convolutional neural network extracts image features by performing convolution operations in a three dimensional space, comprising:
$$( {O}_{i , j , k} ) = {\sum }_{m , n , p , q} {I}_{i + m - 1 , j + n - 1 , k + p - 1} \cdot {W}_{m , n , p , q} + b ;$$
wherein O_{i,j,k} is a pixel value of an output feature map in an ith row, jth column, and kth layer; I_{i+m-1, j+n-1,k+p-1} is a pixel value in an input image cube, at a corresponding position, wherein m, n, and p are offsets of a convolution kernel in each dimension; and W_{m,n,p,q} is a weight of the convolution kernel in a mth row, nth column, pth layer, and qth channel, and b is a bias term.

7. The radiotherapy positioning and dose optimization system based on deep learning and multimodal image fusion based on claim 5, wherein a long and short-term memory network is configured to process sequential data to capture time dependency, comprising:
$${f}_{t} = σ ( {W}_{f} \cdot [ {h}_{t - 1} , {x}_{t} ] + {b}_{f} )$$
$${i}_{t} = σ ( {W}_{i} \cdot [ {h}_{t - 1} , {x}_{t} ] + {b}_{i} )$$
$${o}_{t} = σ ( {W}_{o} \cdot [ {h}_{t - 1} , {x}_{t} ] + {b}_{o} )$$
$${c}_{t} = {f}_{t} ⊙ {c}_{t - 1} + {i}_{t} ⊙ tanh ( {W}_{c} \cdot [ {h}_{t - 1} , {x}_{t} ] + {b}_{c} )$$
$${h}_{t} = {o}_{t} ⊙ tanh ( ct ) ;$$
where fₜ, iₜ, oₜ represent activation values of a forgetting gate, an input gate, and an output gate, respectively, at time step t, which are between 0 and 1, and determine how the information is forgotten, updated, or output; cₜ is a cellular state, which preserves the long-term dependency information from the past to a current time step; hₜ is a hidden state, which is an output of the current time step, and combines forgotten information with new input information; and σ is a sigmoid function for squeezing an input value to between (0, 1) and is commonly used in gating mechanisms; tanh is a hyperbolic tangent function for squeezing the input to (−1, 1) and is commonly used in state updating; W_{f}, Wᵢ, Wₒ, W_{c} are weight matrices corresponding to the forgetting gate, the input gate, the output gate, and a unitary state updating, respectively; b_{f}, bᵢ, bₒ, b_{c} are bias terms corresponding to each gate and the state updating; [hₜ₋₁, xₜ] is a splice vector containing a hidden state hₜ₋₁ of a previous time step and an input xₜ of a current time step; and ⊚ denotes an elementwise multiplication operation, i.e., multiplication of corresponding elements of two same shaped arrays.

8. The radiotherapy positioning and dose optimization system based on deep learning and multimodal image fusion according to claim 5, wherein the adaptive optimization algorithm is configured to optimize network weights, comprising:
$${m}_{t} = {β}_{1} {m}_{t - 1} + ( 1 - {β}_{1} ) {g}_{t}$$
$${v}_{t} = {β}_{2} {v}_{t - 1} + ( 1 - {β}_{2} ) {g}_{t}^{2}$$
$${\hat{m}}_{t} = \frac{{m}_{t}}{1 - {β}_{1}^{t}}$$
$${\hat{v}}_{t} = \frac{{v}_{t}}{1 - {β}_{2}^{t}}$$
$${θ}_{t} = {θ}_{t - 1} - α \frac{{\hat{m}}_{t}}{\sqrt{{\hat{v}}_{t}} + ε}$$
where mₜ, vₜ are a first-order moment estimate (momentum) and a second-order moment estimate (variance) at a tth iteration, respectively; β₁, β₂ are hyperparameters of Adam's optimizer, which control decay rates of the first-order moment estimate and the second-order moment estimate, respectively; gₜ is a gradient at the tth iteration; \hat{m}ₜ, \hat{v}ₜ is a corrected (bias-corrected) first-order moment estimate and second-order moment estimate, which are configured to solve an initial bias problem; α is a learning rate, which determines a step size updated every time; ε is a constant to avoid division by zero.

9. The radiotherapy positioning and dose optimization system based on deep learning and multimodal image fusion based on claim 1, wherein steps of the dose optimization calculation module for optimizing the dose further comprises:
S6, dose distribution evaluation: after the optimization process, evaluating the generated dose distribution, comprising a dose-volume histogram analysis, to evaluate whether a target region and critical organs have achieved a predetermined dosimetric target;
S7, multiple iterations and optimization: the optimization calculation module performing multiple iterations to gradually approximate an ideal dose distribution, wherein each iteration is corrected and refined based on a previous result.

10. The radiotherapy positioning and dose optimization system based on deep learning and multimodal image fusion according to claim 9, wherein the particle swarm optimization algorithm specifically comprises:
velocity update:
$${v}_{id} ( t + 1 ) = w \cdot {v}_{id} ( t ) + {c}_{1} {r}_{1} ( {pbest}_{id} - {x}_{id} ( t ) ) + {c}_{2} {r}_{2} ( gbest - {x}_{id} ( t ) ) ;$$
position update:
$${x}_{id} ( t + 1 ) = {x}_{id} ( t ) + {v}_{id} ( t + 1 ) ;$$
where, v_{id} (t) is a velocity vector of a particle i at the tth iteration, which represents a velocity of the particle i in each dimension of a search space; x_{id} (t) is a position vector of the particle i at the tth iteration, which represents a current position of the particle i in the search space; pbest_{id} is a personal optimal position of the particle i, i.e., an optimal solution found by the particle i during a search process, the gbest is a global optimal position, i.e., a current optimal solution found in a whole swarm; w is an inertia weight for balancing a degree of memory of the particle's own speed and a degree of following the optimal information during the search process, and the inertia weight is gradually reduced with the number of iterations to improve a convergence of the algorithm; c₁ and c₂ are acceleration constants, which control the extent to which the particle adjust its velocity toward an individual optimal solution and a global optimal solution for a group of which the individual is a member; r₁ and r₂ are random numbers, which are randomly generated from [0, 1] at each iteration and are configured to introduce a random factor to avoid the algorithm from converging to the local optimal solution prematurely;
wherein the simulated annealing algorithm specifically comprises:
$$P ( accept ) = min ( 1 , {e}^{- \frac{Δ E}{T}} )$$
$$Δ E = {E}_{new} - {E}_{current} ;$$
where P(accept) denotes a probability of a worse new solution; E_{new} denotes an energy of a new state; E_{current} denotes an energy of a current state; ΔE denotes an energy difference, if ΔE is negative, a new solution is superior to a current solution; if ΔE is positive, the new solution is inferior to the current solution; T denotes a simulated temperature.
